(19) United States
(12) Patent Application Publication
(10) Pub. No.: US 2023/0027192 A1
(43) Pub. Date: Jan. 26, 2023

(54) METHODS OF USING SOLUBLE SOLID DISPERSIONS OF RIFAXIMIN

(71) Applicant: United States Government as Represented by the Department of Veterans Affairs, Washington, DC (US)

(72) Inventor: Jasmohan S. Bajaj, Richmond, VA (US)

(21) Appl. No.: 17/779,865

(22) PCT Filed: Nov. 24, 2020
(86) PCT No.: PCT/US2020/061940

§ 371 Date: May 25, 2022

Related U.S. Application Data
(60) Provisional application No. 62/939,860, filed on Nov. 25, 2019.
(60) Provisional application No. 63/082,155, filed on Sep. 23, 2020.

(51) Int. Cl. A61K 31/437; A61K 9/20; A61K 9/28; A61P 1/14; A61P 1/16

(52) U.S. Cl. CPC A61K 31/437; A61K 9/2054; A61K 9/2031; A61K 9/2009; A61K 9/2013; A61K 9/284; A61P 1/14; A61P 1/16

(57) ABSTRACT

Provided herein are solid dispersions comprising rifaximin and pharmaceutical compositions and uses thereof.

### RELATED APPLICATIONS

This application claims priority to U.S. Provisional Application No. 62/939,860, filed Nov. 25, 2019 and U.S. Provisional Application No. 63/082,155, filed Sep. 23, 2020, the entire contents of each of which are incorporated herein by reference.

### FIELD OF THE INVENTION

The inventions described herein relate generally, but not exclusively, to rifaximin and compositions thereof and uses of the same, including the use to beneficially affect or increase fecal bile acid concentrations in subjects and/or the ratio of secondary to primary fecal bile acids.

### BACKGROUND OF THE INVENTION

There is emerging evidence that the gut milieu plays an important role in the progression of the complications of cirrhosis. J Hepatol. 2013 May; 58(5): 949-955. The complex relationship between cirrhosis, bile acids, and gut microbiota has only recently started being unraveled. Gut Microbes. 2013 September-October; 4(5):382-7.

It has been postulated that bile acid pool size and composition appear to be a major regulator of microbiome structure, which in turn appears to be an important regulator of bile acid pool size and composition, with the balance between the equilibrium being critical for human health and disease. Gut Microbes. 2013 September-October; 4(5):382-7. It is suggested that cirrhosis, especially advanced disease, is associated with a decreased conversion of primary to secondary fecal bile acids which is linked with abundance of key gut microbiome taxa. J Hepatol. 2013 May; 58(5): 949-955. Further studies have been called for to identify bile acids as therapeutic targets for gut bacterial modification to prevent progression and complications of cirrhosis. J Hepatol. 2013 May; 58(5): 949-955.

It has been proposed that in healthy individuals, normal levels of bile salts are metabolized from cholesterol in the neutral bile acid biosynthetic pathway; 7a-dehydroxylating bacteria thrive from energy derived from normal levels of primary bile acids, and the ratio of secondary to primary bile acids is normal. In contrast, it is proposed that in the liver of patients with cirrhosis, inflammation shifts cholesterol metabolism to the acidic pathway, and more of the secondary bile acid deoxycholic acid is produced. It is proposed that the ratio of secondary to primary bile acids is reduced due to lower bile acid synthesis. Ailment Pharmacol. Ther. 2016, 43 (Suppl 1): 11-26.

The role of rifaximin in the management of hepatic encephalopathy (HE) and other complications of cirrhosis has been investigated. Ailment Pharmacol. Ther. 2016, 43 (Suppl 1) 11-26. For example, it has been reported that rifaximin resulted in a reduction of the secondary/primary bile acid ratio in patients with compensated cirrhosis. Ailment Pharmacol. Ther. 2016, 43 (Suppl 1) 11-26; J Hepatol. 2013 May; 58(5): 949-955. The possibility that the bacteria that are normally suppressed by rifaximin could be responsible for secondary bile acid production has been reported; the hypothesis that rifaximin acts in part by changing the behavior of the gut microbiota by reducing the production of secondary bile acids has also been reported. J Hepatol. 2013 May; 58(5): 949-955.

It has additionally been reported that the nonsystemic agent rifaximin reduces the risk of HE recurrence and HE-related hospitalizations in cirrhosis and that in patients with cirrhosis, rifaximin modulates the bacterial composition of the gut microbiota without a consistent effect on overall fecal microbiota composition. Ailment Pharmacol. Ther. 2016, 43 (Suppl 1) 11-26. It has also been reported that certain pharmaceutical compositions comprising solid dispersions of rifaximin effectively reduce the time to hospitalization and prevent all-cause mortality associated with complications of liver disease. U.S. Pat. App'n Pub. No. 2019/0224175 A1.

### BRIEF SUMMARY OF THE INVENTION

It has been surprisingly discovered that certain pharmaceutical compositions comprising rifaximin beneficially affect or increase the total and secondary bile acid concentrations in patients with early decompensated cirrhosis and that certain pharmaceutical compositions comprising rifaximin have a beneficial impact on gut microbial function.

In one aspect, the invention provides a use of pharmaceutical compositions comprising rifaximin to beneficially affect or increase the total and/or secondary fecal bile acid concentrations in a subject. In some embodiments, the compositions comprise rifaximin soluble solid dispersion (SSD) tablets. In some embodiments, the compositions are formulated as SSD immediate release (IR) tablets described in detail in U.S. Pat. App'n Pub. No. 2019/0224175 A1, published Jul. 25, 2019, which is herein incorporated by reference.

In one aspect, a method is provided for treating a subject in need of such treatment with an effective amount of rifaximin. In some embodiments, the method involves administering to the subject an effective amount of rifaximin in the form of an SSD IR tablet according to U.S. Pat. App'n Pub. No. 2019/0224175 A1. In some embodiments, the method includes administering about 40 mg of rifaximin in the form of a rifaximin SSD IR tablet. In some embodiments, the method is a method for beneficially affecting or increasing the total and/or secondary bile acid concentrations in a subject in need thereof, including but not limited to a subject with compromised gut microbial function, irritable bowel syndrome, diabetes, obesity, small intestinal bacterial overgrowth, alcoholism, colon cancer and adenomas, non-alcoholic fatty liver, cirrhosis (including but not limited to, for example, compensated and decompensated cirrhosis, including but not limited to early decompensated cirrhosis), and/or complications of cirrhosis such as, e.g., hepatic encephalopathy (HE), esophageal variceal bleeding (EVB), spontaneous bacterial peritonitis (SBP), and hepatorenal syndrome (HRS).

### BRIEF DESCRIPTION OF THE DRAWINGS

The foregoing summary, as well as the following detailed description of the invention, will be better understood when read in conjunction with the appended FIGURES.

FIG. 1 represents box plots of bile acid concentrations over time. As shown therein, the data represent log transformed values (μmol/g of stool sample) of bile acid concentrations reported for Total Bile Acids, Primary Bile Acids, and Secondary Bile Acids. Outliers are represented by a diamond symbol. IR=immediate release. SSD=soluble solid dispersion.

### DETAILED DESCRIPTION OF THE INVENTION

It has been surprisingly discovered that modulating gut microbiota dysbiosis using a nonsystemic antibiotic such as rifaximin may beneficially impact the fecal bile acid pool and affects or increase total and/or secondary fecal bile acid concentrations. Indeed, it has been surprisingly discovered that for patients with early decompensated cirrhosis treated with rifaximin SSD tablets, which has improved water solubility and moderate systemic exposure, beneficially impacts the fecal bile acid pool and affects or increases total and/or secondary fecal bile acid concentrations.

Unless defined otherwise, all technical and scientific terms used herein have the same meaning as is commonly understood by a person having ordinary skill in the art to which this invention pertains. All patents and publications referred to herein are incorporated by reference in their entireties.

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The language “effective amount” refers to an amount of rifaximin (e.g., a composition comprising a solid dispersion of rifaximin) effective, upon single or multiple dose administration to the subject that is sufficient to affect the intended application. An effective amount may vary depending upon the intended application, or the subject and disease condition being treated (e.g., the weight, age and gender of the subject), the severity of the disease condition, the manner of administration, etc, which can readily be determined by one of ordinary skill in the art. The specific may will vary depending on the particular compounds chosen, the dosing regimen to be followed, whether the compound is administered in combination with other compounds, timing of administration, the tissue to which it is administered, and the physical delivery system in which the compound is carried.

The language “beneficially affect or increase” refers to changing or increasing the amount of bile acids in a subject. The change or increase may vary depending upon the intended application, or the subject and disease condition being treated (e.g., the weight, age and gender of the subject), the severity of the disease condition, the manner of administration, etc, which can readily be determined by one of ordinary skill in the art. Further, it is understood that measuring the amount of bile acids in a subject can readily be determined by one of ordinary skill in the art. Bile acids can be extracted from stool and profiles can be determined using high performance liquid chromatography, per published methods (Kakiyama G, et al. J Lipid Res. 2014; 55 [5]:978-990).

As used herein, the terms “treat,” “treatment,” and/or “treating” may refer to the management of a disease, disorder, or pathological condition (e.g., an disease or condition described herein), or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition). In one aspect, treatment refers to therapeutic treatment.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

In the present disclosure, when a numerical value is modified by the term “about”, the exact numerical value is also deemed to be disclosed. Use of the term “about” when referring to a number or a numerical range means that the number or numerical range referred to is an approximation and thus the number or numerical range may vary.

Furthermore, the transitional terms “comprising”, “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments described herein that encompass the invention can, in alternate embodiments, be more specifically defined by any of the transitional terms “comprising,” “consisting essentially of,” and “consisting of.”

In one embodiment, the invention provides a use of pharmaceutical compositions comprising rifaximin to beneficially affect or increase the total and/or secondary fecal bile acid concentrations in a subject. In an embodiment, the compositions are for oral administration. In one embodiment, the compositions comprise rifaximin SSD tablets for oral administration. In one embodiment, the compositions comprise rifaximin SSD IR tablets for oral administration. In certain embodiments, the compositions are formulated as SSD tablets for oral administration described in detail in U.S. Pat. App'n Pub. No. 2019/0224175 A1. In one embodiment, such compositions are formulated as an SSD IR tablet comprising 40 mg rifaximin and hydroxypropyl methylcellulose acetate succinate (HPMC-AS). In one embodiment, such compositions comprise the formula of Example 1 herein. Rifaximin SSD tablet formulations are set forth, for example, in U.S. Pat. No. 9,737,610 and U.S. Pat. App'n Pub. No. 2019/0224175, the entirety of which are incorporated herein by reference.

Pharmaceutical compositions of the compounds described herein may also be prepared from a composition described herein and one or more pharmaceutically acceptable excipients suitable for sublingual, buccal, rectal, intraosseous, intraocular, intranasal, epidural, or intrathecal administration.

The compositions described herein can be used in methods for treating disease. In one embodiment, a method is provided for treating a subject in need of such treatment with an effective amount of rifaximin. The method involves administering to the subject an effective amount of rifaximin, preferably in the form of an SSD IR 40 mg tablet according to U.S. Pat. App'n Pub. No. 2019/0224175 A1. In one embodiment, the method is a method for beneficially affecting or increasing the total and/or secondary fecal bile acid concentrations in a subject in need thereof, including but not limited to a subject with compromised gut microbial function, diabetes, irritable bowel syndrome, obesity, small intestinal bacterial overgrowth, alcohol intake, alcoholism, colon cancer and adenomas, non-alcoholic fatty liver, cirrhosis (including but not limited to, for example, compensated and decompensated cirrhosis, including but not limited to early decompensated cirrhosis), and/or complications of cirrhosis such as, e.g., hepatic encephalopathy (HE), esophageal variceal bleeding (EVB), spontaneous bacterial peritonitis (SBP), and hepatorenal syndrome (HRS).

In some embodiments of the methods described herein, the composition described herein is administered for at least 24 weeks. In some embodiments, the composition described herein is administered for about 24 weeks. In some embodiments, the composition described herein is administered for at most 24 weeks. In some embodiments, the composition described herein is administered to subject, QD, BID, TID, or QID. In some embodiments, the composition described herein is administered QD.

In some embodiments of the methods described herein, a patient may demonstrate an increase in total fecal bile acid concentrations within about 4 weeks. In some embodiments, the increase in total fecal bile acid concentrations for the patient may be about 0.1, about 0.2, about 0.3, about 0.4, about 0.5, about 0.6, about 0.7, about 0.8, about 0.9, about 1.0, about 1.1, about 1.2, about 1.3, about 1.4, or about 1.5 μmol/g of stool sample. In some embodiments, the increase in total fecal bile acid concentrations for the patient may be at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, or at least 1.5 μmol/g of stool sample. In some embodiments, the increase in total fecal bile acid concentrations for the patient may be at most 0.1, at most 0.2, at most 0.3, at most 0.4, at most 0.5, at most 0.6, at most 0.7, at most 0.8, at most 0.9, at most 1.0, at most 1.1, at most 1.2, at most 1.3, at most 1.4, or at most 1.5 μmol/g of stool sample. In some embodiments, the increase in total fecal bile acid concentrations for the patient may be about 0.01 to about 1.5, or about 0.01 to about 1.4, or about 0.01 to about 1.3, or about 0.01 to about 1.2, or about 0.01 to about 1.1, or about 0.01 to about 1.0 μmol/g of stool sample. In some embodiments, the increase in total fecal bile acid concentrations for the patient may be about 0.1 to about 1.5, or about 0.1 to about 1.4, or about 0.1 to about 1.3, or about 0.1 to about 1.2, or about 0.1 to about 1.1, or about 0.1 to about 1.0 μmol/g of stool sample. In some embodiments, the increase in total fecal bile acid concentrations for the patient may be about 0.6 μmol/g of stool sample.

In some embodiments of the methods described herein, a patient may demonstrate an increase in secondary fecal bile acid concentrations within about 4 weeks. In some embodiments, the increase in secondary fecal bile acid concentrations for the patient may be about 0.1, about 0.2, about 0.3, about 0.4, about 0.5, about 0.6, about 0.7, about 0.8, about 0.9, about 1.0, about 1.1, about 1.2, about 1.3, about 1.4, or about 1.5 μmol/g of stool sample. In some embodiments, the increase in secondary fecal bile acid concentrations for the patient may be at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, or at least 1.5 μmol/g of stool sample. In some embodiments, the increase in secondary fecal bile acid concentrations for the patient may be at most 0.1, at most 0.2, at most 0.3, at most 0.4, at most 0.5, at most 0.6, at most 0.7, at most 0.8, at most 0.9, at most 1.0, at most 1.1, at most 1.2, at most 1.3, at most 1.4, or at most 1.5 μmol/g of stool sample. In some embodiments, the increase in secondary fecal bile acid concentrations for the patient may be about 0.01 to about 2.0, or about 0.01 to about 1.9, or about 0.01 to about 1.8, 0.01 to about 1.7, or about 0.01 to about 1.6, or about 0.01 to about 1.5, or about 0.01 to about 1.4, or about 0.01 to about 1.3, or about 0.01 to about 1.2, or about 0.01 to about 1.1, or about 0.01 to about 1.0 μmol/g of stool sample. In some embodiments, the increase in secondary fecal bile acid concentrations for the patient may be about 0.1 to about 1.5, or about 0.1 to about 1.4, or about 0.1 to about 1.3, or about 0.1 to about 1.2, or about 0.1 to about 1.1, or about 0.1 to about 1.0 μmol/g of stool sample. In some embodiments, the increase in secondary bile acid concentrations may about 0.9 μmol/g of stool sample.

While certain embodiments of the invention have been described and/or exemplified above, various other embodiments will be apparent to those skilled in the art from the foregoing disclosure. The invention is, therefore, not limited to the particular embodiments described and/or exemplified, but is capable of considerable variation and modification without departure from the scope and spirit of the appended claims.

### EXAMPLES

The following examples describe the invention in further detail. These examples are provided for illustrative purposes only and should in no way be considered as limiting the invention.

### Example 1

The components and composition of a 40 mg SSD immediate-release (IR) tablet is provided in Table 1 below, as described in U.S. Pat. App'n Pub. No. 2019/0224175 A1.

| | | Theoretical Quantity |
|---|---|---|
| Ingredient | Function | (mg/tablet) |
| Rifaximin | Active | 40 |
| HPMC-AS | Polymer | 40 |
| Poloxamer 407 | Surfactant | 4.75 |
| Croscarmellose | Dissolution | 20.74 |
| sodium | enhancer | |
| Microcrystalline | Filler | 119.43 |
| cellulose | | |
| Colloidal silicon | Glidant | 0.45 |
| dioxide | | |
| Magnesium stearate | Lubricant | 1.13 |
| (non-bovine) | | |
| Opadry II Blue | Coating | 11.92 |
| 85F90614 | | |
| (PVA coating) | | |
| Purified Water | Solvent for | — |
| | coating solution | |

| | |
|---|---|
| Total Theoretical Weight | 238.42 |

### Example 2

Methods: In a phase 2, randomized, double-blind, placebo-controlled, dose-finding trial, adults with cirrhosis and ascites (grade ˜l), with no history of esophageal variceal bleeding or spontaneous bacterial peritonitis, received rifaximin SSD or placebo once nightly for 24 weeks (Clinicaltrials.gov identifier: NCT01904409). This post hoc subanalysis evaluated data from patients receiving rifaximin SSD IR tablets 40 mg (most efficacious dose) or placebo once daily. Stool samples were collected at screening (Day −21 to Day −7), after 24 weeks of treatment, and at 2 weeks post-treatment (Week 26). Bile acids were extracted from stool and profiles were determined using high performance liquid chromatography, per published methods (Kakiyama G, et al. J Lipid Res. 2014; 55 [5]:978-990).

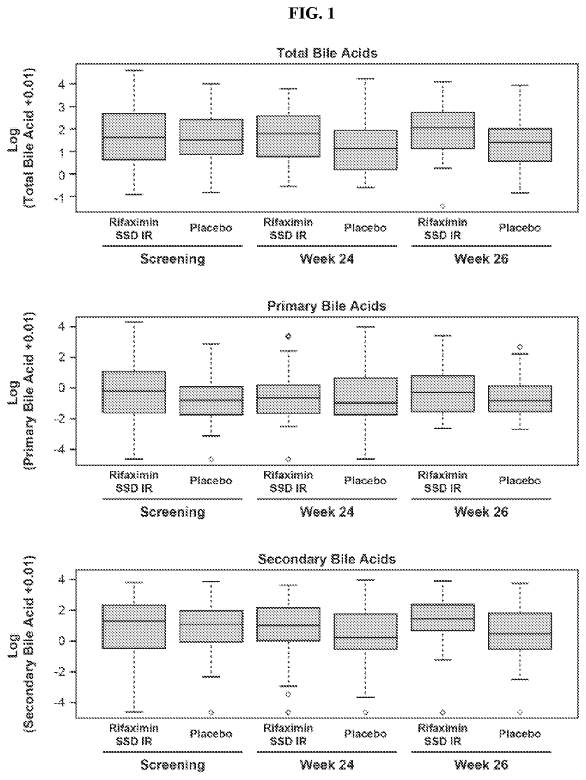

Results and conclusions: Stool samples were analyzed for 48 patients in rifaximin SSD IR 40-mg group and 50 patients in the placebo group. At screening, mean (SD) total fecal bile acid concentrations were 12.2 (18.6) and 8.2 (9.8) μmol/g of stool for rifaximin SSD IR 40 mg and placebo, respectively; 3.8 (10.8) and 1.6 (3.2) μmol/g of stool for primary bile acids; and 7.7 (10.2) and 5.7 (8.4) μmol/g of stool sample for secondary bile acids. A significantly higher mean concentration of total and secondary bile acids were observed in the rifaximin SSD IR 40-mg group versus placebo at Weeks 24 and 26 (FIG. 1): for total bile acids, the difference was 0.6 μmol/g (95% CI, 0.1-1.1; P=0.02), and for secondary bile acids, 0.9 μmol/g (95% CI, 0.0-1.7; P=0.046).

Rifaximin SSD IR 40 mg once daily for 24 weeks increased the total and secondary fecal bile acid concentrations versus placebo in patients with early decompensated cirrhosis. This indicates that rifaximin SSD IR treatment has an impact on gut microbial function.

1-24. (canceled)

25. A method of increasing total fecal bile acid concentrations in a subject, comprising administering to said subject a soluble solid dispersion tablet comprising rifaximin, wherein after said administration to said patient, the total fecal bile acid concentrations increase within about 4 weeks.

26. The method of claim 25, wherein the tablet comprises about 40 mg rifaximin.

27. The method of claim 25, wherein the tablet further comprises HPMC-AS.

28. The method of claim 25, wherein the tablet comprises about 40 mg rifaximin and about 40 mg HPMC-AS.

29. The method of claim 25, wherein the total fecal bile acid concentrations increase by about 0.1-1.1 μmol/g.

30. The method of claim 29, wherein the total fecal bile acid concentrations increase by about 0.6 μmol/g.

31. A method of increasing secondary fecal bile acid concentrations in a subject, comprising administering to said subject a soluble solid dispersion tablet comprising rifaximin, wherein after said administration to said patient, the secondary fecal bile acid concentrations increase within about 4 weeks.

32. The method of claim 31, wherein the tablet comprises about 40 mg rifaximin.

33. The method of claim 31, wherein the tablet further comprises HPMC-AS.

34. The method of claim 31, wherein the tablet comprises about 40 mg rifaximin and about 40 mg HPMC-AS.

35. The method of claim 31, wherein the secondary fecal bile acid concentrations increase by about 0.01-1.7 μmol/g.

36. The method of claim 35, wherein the secondary fecal bile acid concentrations increase by about 0.9 μmol/g.

37. The method of claim 25 or 35, wherein the subject has early decompensated cirrhosis.

38. A method of treating a disease in a subject in need thereof, wherein treatment of the disease is effected by increasing the total and/or secondary bile acid concentrations in the subject, the method comprising administering a therapeutically effective dose of rifaximin to the subject in unit dosage form, wherein the unit dosage form consists of an immediate release soluble solid dispersion (SSD) tablet.

39. The method of claim 38, wherein the therapeutically effective dose of rifaximin is 40 mg of rifaximin.

40. The method of claim 38, wherein the rifaximin is delivered once daily to the subject.

41. The method of claim 38, wherein the rifaximin is delivered once daily to the subject for 24 weeks.

42. The method of claim 38, wherein the disease is selected from the group consisting of compromised gut microbial function, diabetes, irritable bowel syndrome, obesity, small intestinal bacterial overgrowth, alcoholism, colon cancer and adenomas, non-alcoholic fatty liver, cirrhosis, hepatic encephalopathy (HE), esophageal variceal bleeding (EVB), spontaneous bacterial peritonitis (SBP), and hepatorenal syndrome (HRS).

43. The method of claim 42, wherein the disease is selected from the group consisting of obesity, alcoholism, esophageal variceal bleeding (EVB), spontaneous bacterial peritonitis (SBP), and hepatorenal syndrome (HRS).

44. The method of claim 43, wherein the disease is obesity or alcoholism.
